(19) United States
(12) Patent Application Publication
(10) Pub. No.: US 2015/0351934 A1
(43) Pub. Date: Dec. 10, 2015

(54) PROSTHESIS ELEMENT AND METHOD FOR PRODUCING A PROSTHESIS ELEMENT

(71) Applicant: OTTO BOCK HEALTHCARE GMBH, Duderstadt (DE)

(72) Inventors: Steffen SAWATZKI, Sollstedt (DE); Markus SCHNEEGANS, Rollshausen (DE); Sven ZARLING, Duderstadt (DE); Sandra NIEDERSTRASSER, Duderstadt (DE); Andreas KRUSE, Worbis (DE); Ilka SCHLESIGER, Jena (DE); Martin STOHR, Jena (DE); Dirk HAASE, Jena (DE); Esther GLASKER, Jena (DE)

(21) Appl. No.: 14/759,923

(22) PCT Filed: Jan. 17, 2014
(86) PCT No.: PCT/EP2014/000118

§ 371 Date: Jul. 8, 2015

(30) Foreign Application Priority Data
Jan. 18, 2013 (DE) 10 2013 000 770.4

(51) Int. Cl. A61F 2/50; A61F 2/78

(52) U.S. Cl. CPC A61F 2/5046; A61F 2/5044; A61F 2/7812; A61F 2002/5001; A61F 2002/5038; A61F 2002/7806

(57) ABSTRACT

A prosthesis element for lining a prosthesis or for forming a prosthesis component and to a method for producing a prosthesis element from a flat base material, consisting of at least one blank made of a flat base material. The blank is bent, whereby a hollow body is formed, opposing edges being connected to one another in the bent state.

The invention relates to a prosthesis element for lining a prosthesis or for forming a prosthesis component and to a method for producing a prosthesis element from a flat base material.

Prostheses replace missing limbs from a functional and cosmetic viewpoint. They generally consist of a number of elements, which interact with one another to meet the requirements expected of them. Apart from a device for attachment to the user of the prosthesis, for example in the form of a socket into which a remaining part of a limb is inserted, articulating devices or distally arranged end elements are provided for example. When providing a prosthesis for a patient who has an upper leg stump, the usual components are an upper leg socket, a prosthetic knee joint, a lower leg tube and a prosthetic foot. In the case of patients who have an upper arm stump, an upper arm socket, an elbow joint, a lower arm tube and a prosthetic hand with a prosthetic wrist are provided. These components are generally designed on the basis of functional aspects and are not formed in such a way as to approximate the natural shape. In particular, the articulating devices and the devices in between, that is to say the lower leg tube and the lower arm tube, are designed as purely functional elements in the form of a tube, which is unsatisfactory from esthetic aspects.

DE 299 579 B relates to an artificial leg, which is fastened to the upper leg stump by means of a sleeve. There is an internal skeleton, which connects the sleeve to an prosthetic foot mounted in an articulated manner. An elastic sheath is arranged around the lower leg.

DE 622 314 B relates to an artificial leg for upper leg amputees, with a sleeve receiving the upper leg stump and a lower leg, comprising two tubes sliding one inside the other, articulated thereto by means of a fork. A cosmetic cover not designated any more specifically is arranged around the mechanical devices.

U.S. Pat. No. 3,545,009 A relates to an upper leg socket which can be set in its circumference. The socket may be formed from wood or plastic or a fiber-reinforced plastic and has slots, so that the circumference can be varied by way of a toggle clamp.

DE 285917 B1 relates to an artificial knee joint with adjoining lower leg sleeve with a piece of leather enclosing the knee, which is partially pleated. The piece of leather is connected to the lower leg sleeve, which consists of two sleeves that are pushed one inside the other and are provided with toothed racks. The toothed racks are mounted resiliently in relation to one another, so that they keep the sleeves in the correct position in relation to one another for use. The sleeve consists of an inner piece, which consists of a number of steel strips lying next to one another in parallel, which are embedded in a woven fabric or other material. Arranged over the inner sleeve is a thin-walled steel sleeve, which bears a toothing opposite the toothed rack. The steel sleeve is covered by a wooden sleeve, which advantageously consists of pieces of plywood placed crosswise one over the other.

The object of the present invention is to provide a prosthesis element for lining a prosthesis or for forming a prosthesis component and a method for producing a prosthesis element with which low-cost production and easy adaptability to the respective user of the prosthesis are possible.

This object is achieved according to the invention by a prosthesis element with the features of the main claim and a method with the features of the independent claim. Advantageous configurations and developments of the invention are presented in the respective subclaims.

The prosthesis element for lining a prosthesis or for forming a prosthesis component consists of at least one blank of a flat base material, which is bent to form a hollow body, opposing edges in the bent state being connected to one another. While cosmetic linings for prostheses of the lower extremities must at present be produced from a preformed foam by an orthopedic technician, who must mill away superfluous material for adaptation to the desired anatomical contour, the prosthesis element according to the invention envisages the production of a three-dimensional cosmetic lining of a prosthesis or a three-dimensional prosthesis component that is cut to size. The cut-to-size blank of this material is then shaped into a three-dimensional prosthesis element to form a hollow space and is permanently connected at the opposing edges. The edges that are regarded as opposing edges are those edges of the blank that are opposite one another in the bent state, so that the desired form and contour of the prosthesis element is fixed. Apart from a purely cosmetic application, the prosthesis element also performs functional tasks, for example transfers forces, and consequently forms and replaces functional components of a prosthesis, for example a lower leg tube on a lower arm tube. The prosthesis element is suitable in particular when providing a prosthesis for the first time.

The base material may consist of a foam, a 3D knit or a plastic; it is likewise possible that a metal is used to create an appropriate blank from the base material.

A fiber-reinforced plastic may also be used as the plastic if a permanent and/or force-transferring configuration of the prosthesis element is desired. In the case of a configuration of the base material in the form of a foam or a 3D knit, particularly easy production of the prosthesis element is ensured, and similarly the prosthesis element is particularly lightweight and can be produced at low cost.

Apart from a one-piece configuration of the blank, it is possible that a number of blanks are produced from the base material and, in the end state of the hollow body, opposing edges also of different blanks are connected to one another.

The blank or the blanks may be adhesively bonded and/or welded together at the respectively corresponding, opposing edges, in order to achieve a permanent connection of the opposing, corresponding edges to one another.

Apart from a material-bonding and frictional connection, it is possible that there are arranged or formed on the blank or the blanks form-fitting elements, by way of which the respective edges can be secured to one another, so that in the connected state the hollow body forms. The form-fitting elements may be formed on a separate component and prevent or hinder displacement of the edges in relation to one another in at least one direction. Similarly, the form-fitting elements may be part of the blank, for example in the form of corresponding cutouts or clearances.

One configuration of the invention provides that the blank has a proximal end edge and a distal end edge, the designations proximal and distal referring to the finished, fitted state of the prosthetic element. From the distal end edge there extends at least one cutout in the proximal direction, so that circumferential changes can be brought about at the distal end edge. Similarly, it is additionally or alternatively provided that from the proximal end edge there extends at least one cutout in the distal direction, in order that a curved form can be brought about there. This is advantageous in particular in the case of prosthesis elements that are used as a cosmetic lower leg cover, in order that the constrictions and rounded portions in the ankle area and in the knee area can be realistically replicated. The respective cutouts from the edges into the interior of the blank preferably taper away from the respective end edge, so that an inward curving in the direction of the interior of the hollow body is obtained after the connection of the edges of the cutout in the formation of the hollow body.

Devices for the securement of the prosthesis elements on further prosthesis components may be arranged or formed at a distal or proximal end edge, for example devices for securement on a receiving socket for a stump may be formed at the proximal end, while there are receiving devices for further prosthesis components, for example prosthetic hands or prosthetic feet, at the distal end.

The hollow body formed from the blank is preferably of a cross-sectionally closed form, in order not to allow openings that appear unnatural to be produced within the hollow body.

A number of cutting lines for blanks of different sizes may be provided on the base material, so that, in a way similar to a dressmaking pattern, an orthopedic technician can already choose the blank to be used for different sizes after carrying out a first measurement on the patient. An individual adaptation to the respective patient, in particular to resemble a limb that is not provided with a prosthesis, is still possible.

The prosthesis element may be formed as a cosmetic prosthesis cover or as a load-bearing structural component, for example as a connecting device between a prosthetic foot and a prosthetic knee joint.

The method for producing a prosthesis element on the flat base material provides the steps of cutting out the blank from the flat base material. The cutting out may be configured in various ways, for example by using shears, a laser or by means of water jet cutting. The blank is subsequently bent to form a hollow body, so that edges of the blank are opposite one another. The opposing edges after the bending of the blank are then connected to one another, so that a substantially closed hollow body is formed, usually with openings for the insertion of prosthesis components of the distal and proximal ends.

A development of the invention provides that the blank is bent around the prosthesis and the edges are subsequently connected to one another. As a result, it is possible in a configuration of the prosthesis element as a cosmetic cover to make the functional components of the prosthesis initially match one another and subsequently lay the prosthesis element on the ready-constructed prosthesis.

The blank may be cut out along cutting lines previously provided on the base material, so that, instead of a free-hand configuration of the blank, the mechanical orthopedic engineer is provided with a prefabricated form as a guideline that he can use as a basis. The cutting lines are advantageous in particular when there are receptacles for connection elements to other prosthesis components, so that the exact position of the respective cutouts is established and the production of the prosthesis element is made much easier.

Apart from a permanent connection of the corresponding, opposing edges, it is possible that they are also reversibly connected to one another, so that, in the case where the prosthesis element according to the invention is only provided temporarily, easy removal is possible. The reversible connection may take place by one or more form-fitting elements that are separate or are formed in the blank. As an alternative to this, the edges are preferably adhesively bonded or welded, in order to ensure a permanent assignment of the edges to one another.

A number of blanks may be connected to one another to form the hollow body if the form of the prosthesis element is too complex to achieve the desired hollow body contour.

The prosthesis element may be used for a prosthesis device with a prosthetic foot and an upper leg for bridging a free space that is present between the prosthetic foot and the upper leg and be formed from a blank of the flexible base material. The blank forms a main body, which comprises a hollow space for receiving the prosthesis device, at least one stiffening element being arranged on the main body to increase the intrinsic stability of the prosthesis element. The prosthesis element may be formed as a cuff, for example an ankle cuff, and be used for bridging a free space or transition between two prosthesis components, for example the upper edge or the proximal end of the prosthetic foot and a lower leg part, for example a housing of a damping and controlling device or some other component replicating the volume of a natural lower leg and/or performing the function of a lower leg tube. Other areas of use are likewise possible, for example cosmetic lower leg covers, coverings on upper or lower extremities and load-dissipating devices on prostheses. In the fitted state, the main body comprising the blank forms a hollow space in which the prosthesis device, for example part of the lower leg part, is arranged. The main body may consequently surround a distal portion of the lower leg part in the region of the transition to the prosthetic foot; in the case of other areas of application, other free spaces are bridged. On the main body, a stiffening element is arranged, for example adhesively attached, welded on, molded on or formed on, or fastened in some other way, in order on the one hand to create an appearance that corresponds to the natural limbs, for example the ankle area or distal lower leg area, and on the other hand to provide a functional covering of the mechanical components of the prosthesis device. The stiffening element makes it possible that the prosthesis element has sufficient dimensional stability during movement of the prosthesis device, without the mobility being restricted too much. Similarly, adequate deformability is ensured by the flexible main body, so that furthermore a natural impression is given. The stiffening element makes it possible to adapt the functionality of the prosthesis element to the desired properties, to be specific that there is sufficiently great stability to prevent slipping down or slipping off, or fundamentally to provide sufficient stiffness without losing flexibility that is required to avoid folding or collapsing of the main body, as would be the case with buckling of a rigid material, for instance that of a rubber boot.

The stiffening element in this case consists of a material that has a greater deformation resistance than the base material or the material of the main body, so that the desired dimensional stability and the desired deformation resistance can be achieved or increased specifically at those points at which the stiffening element is arranged. It is similarly possible by the stiffening element to provide an improved mechanical protective effect at particularly sensitive points of the prosthesis, for example at the transition between a prosthetic foot and a lower leg part or in the region of an elbow joint.

The base material may be produced from a textile and/or a foam or comprise a textile and/or a foam and further materials where necessary. Textiles and foams have the advantage of being easy to produce and process and being lightweight while having sufficient flexibility and deformability. The textiles may be formed as woven or knitted fabrics and as knitted spacer fabrics; as foams, both open-cell and closed-cell foams are envisaged. The base material may have a coating, which increases or establishes the water impermeability of the prosthesis element.

For the form-fitting securement of the prosthesis element on a prosthesis component, for example on a prosthetic foot, an upper leg socket, a lower leg part or a lower arm socket, fastening elements may be arranged on the base material or the stiffening element, so that a relative movement between the prosthesis element and a further prosthesis component on which the prosthesis element is secured cannot take place while the prosthesis is being used in the usual way. As a result, it is possible to form or orient the prosthesis element so that it is flush, or virtually flush, with the respective connection element or connection area of the prosthesis component. The fastening elements for the form-fitting securement also bring about the effect that the contour of the prosthesis element adapts itself to the contour of the prosthesis component, so that it is possible to combine different forms or sizes of prosthesis components with one and the same prosthesis element.

A development of the invention provides that the stiffening element is formed as a frame which surrounds the blank or the base material that forms the main body. The frame may be arranged peripherally around the entire circumference of the main body, so that there is sufficient dimensional stability for mounting the prosthesis element, for example on a prosthetic foot or on a lower leg part. The hollow space that is surrounded by the main body is defined by the frame, so that the main body itself may be formed from a limp material which is kept in the desired form by the stiffening element.

At least one hinge device may be arranged in the stiffening element to facilitate flexion about a joint. This hinge device facilitates the relative movement between two prosthesis components, for example the prosthetic foot and the lower leg part, and prevents noises from being produced during flexing due to folding of the prosthesis element. Furthermore, the extraction forces that occur during flexion about a joint are reduced. Reducing the extraction forces ensures that the prosthesis element remains in the intended position in relation to the prosthesis components.

The prosthesis element is advantageously symmetrically formed, the symmetry preferably being in relation to the sagittal plane, so that it is suitable for use both on a left-hand prosthesis device and on a right-hand prosthesis device.

The stiffening element may form the distal and/or proximal end of the prosthesis element, at least parts of the stiffening element forming the distal and/or proximal end. As a result, it is possible that particularly fastening elements, which of course must have a greater strength than the limp or flexible material of the main body, can be produced and formed on more easily. In the case of a separate configuration of the fastening elements, it is possible by the arrangement at the distal and/or proximal end of the prosthesis element to achieve an easy assignment of the fastening element both to the prosthesis element and to the respective prosthesis component. The fastening elements may in this case be secured in a form-fitting manner on the prosthesis element at a form-fitting element, the form-fitting element being formed on or fastened to the stiffening element.

A friction-reducing coating may be arranged on the inner side, in particular in the proximal region, of the base material, and consequently of the main body, and similarly friction-reducing elements may be arranged on the inner side of the base material, for example adhesively attached, molded on, welded on or fastened in some other way to the base material. A friction-reducing coating makes it possible to allow a relative movement between the prosthesis element and the prosthesis component without high extraction forces acting on the prosthesis element, for example ankle cuff, so that a fixed assignment to the prosthesis component is retained, for example between the distal end of the ankle cuff and the proximal end of the prosthetic foot.

The stiffening element may have a closed cross section, in order to increase the dimensional stability. The form is generally oval or made to approximate an oval.

The stiffening element may extend over a relatively great range of height in the proximal-distal extent, it being possible for cutouts to be provided in the stiffening element, in particular in anterior and/or posterior orientation. These cutouts make it possible to allow a deformation of the base material. For this purpose, it is provided that the stiffening element is provided on the base material or main body, so that the cutouts possibly provided in the stiffening element are covered by the base material. Consequently, the prosthesis element is preferably closed in the fitted state, so that no moisture and/or dirt can penetrate.

It may similarly be provided that the main body has an open cross section and the stiffening element is also or only arranged along the opening and closes the cross section, that is to say receives and fixes the opposing edges of the blank of the main body and altogether provides a closed cross section for the prosthesis element.

Exemplary embodiments of the invention are explained in more detail below on the basis of the accompanying figures, in which:

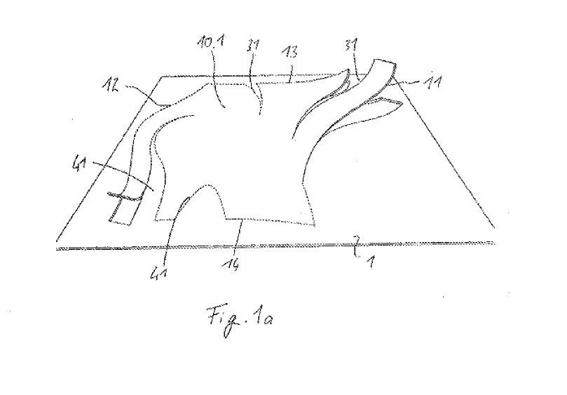

FIG. 1a shows a base material with a partly cut-out blank;

FIG. 1b shows a blank according to Figure la in the partially bent state;

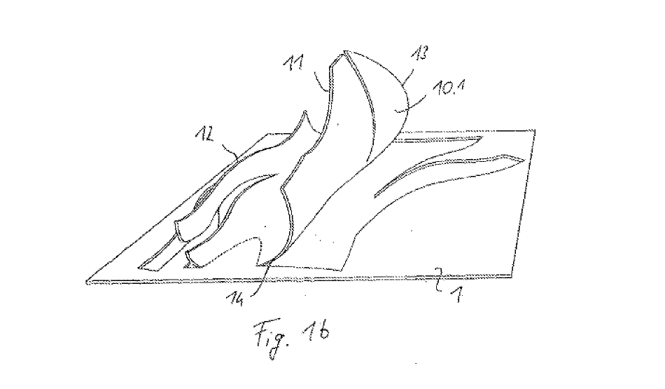

FIG. 2 shows a blank for a cosmetic upper leg cover;

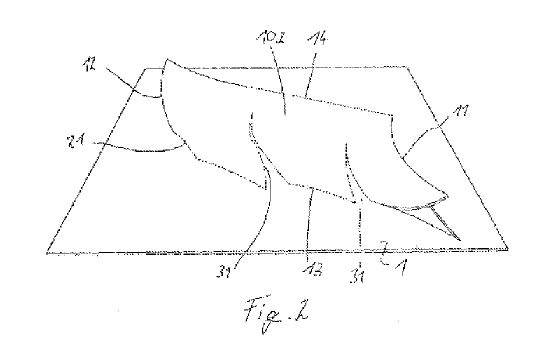

FIG. 3 shows a blank for a foot connection;

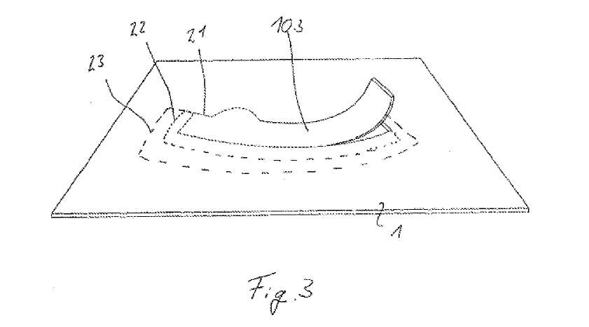

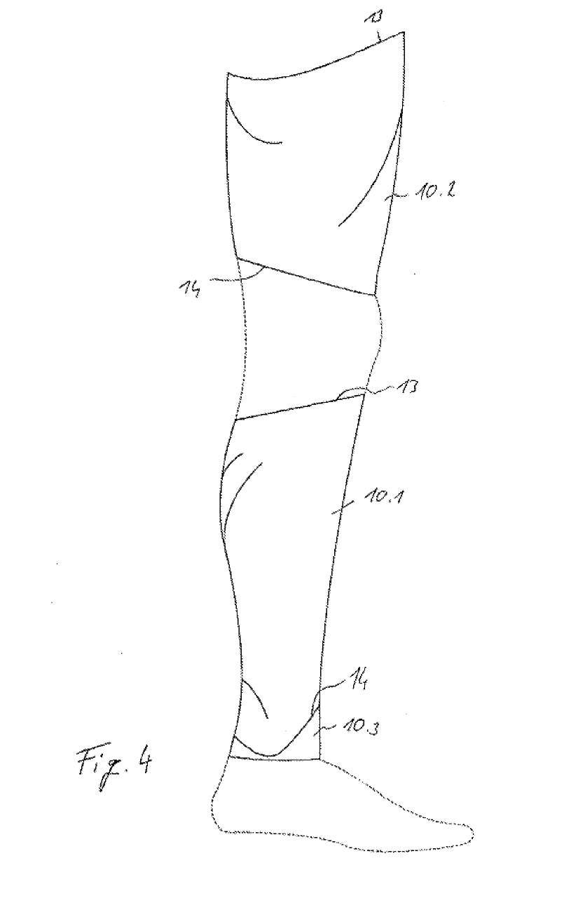

FIG. 4 shows finished prosthesis elements;

FIG. 5 shows a variant of FIG. 1b with a form-fitting element;

FIG. 6 shows a sectional view of a detail of a join;

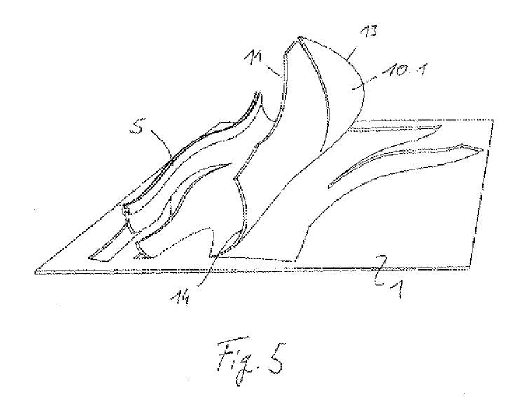

FIG. 7 shows a variant of FIG. 6;

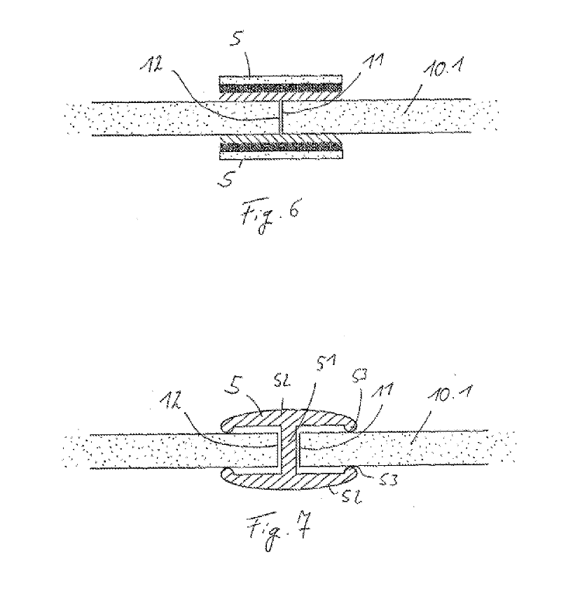

FIG. 8 shows a side view of a prosthesis device with an ankle cuff;

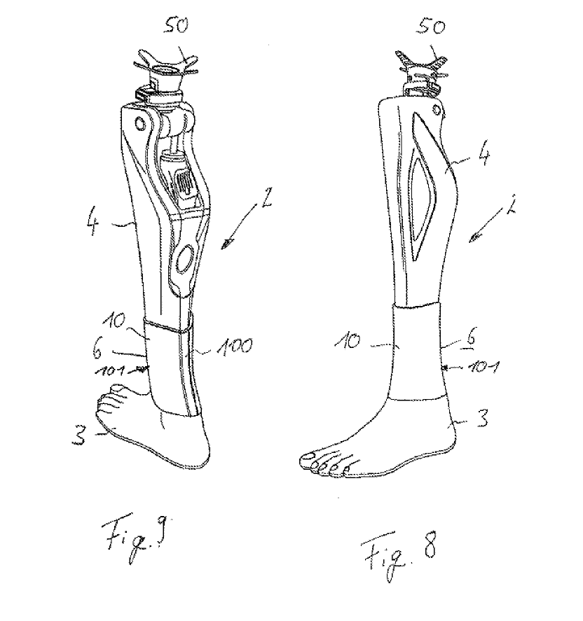

FIG. 9 shows a view of an ankle cuff obliquely from the rear;

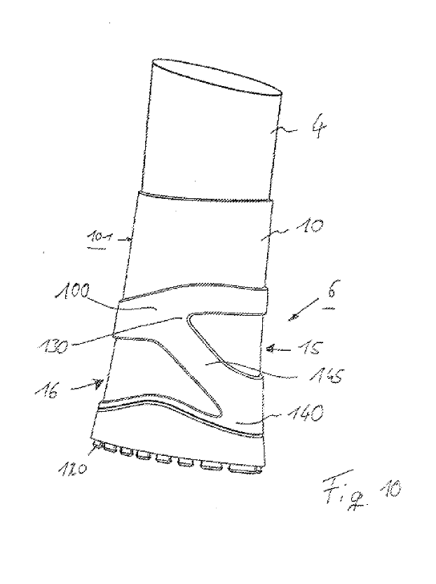

FIG. 10 shows an individual representation of a variant of an ankle cuff;

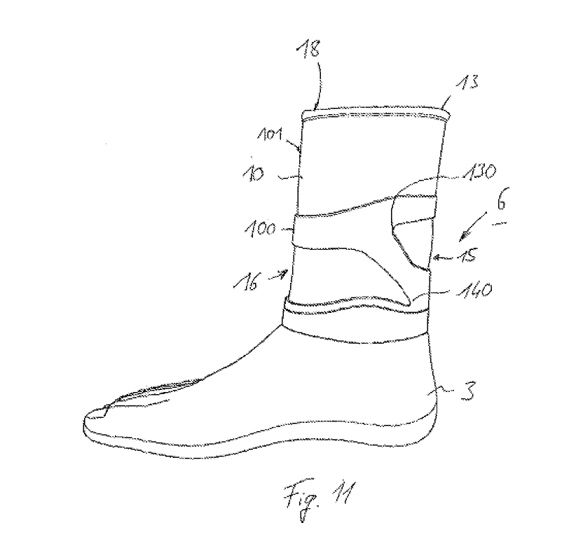

FIG. 11 shows a side view according to FIG. 10;

FIG. 12 shows a rear view according to FIG. 10;

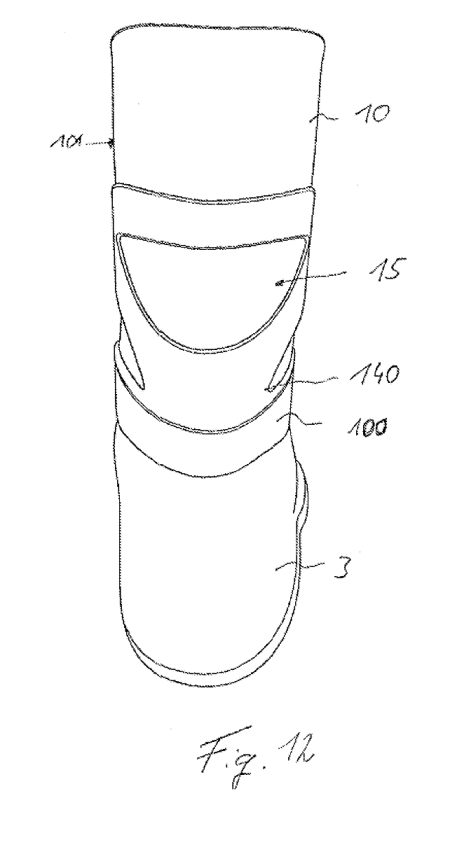

FIG. 13 shows a frontal view according to FIG. 10;

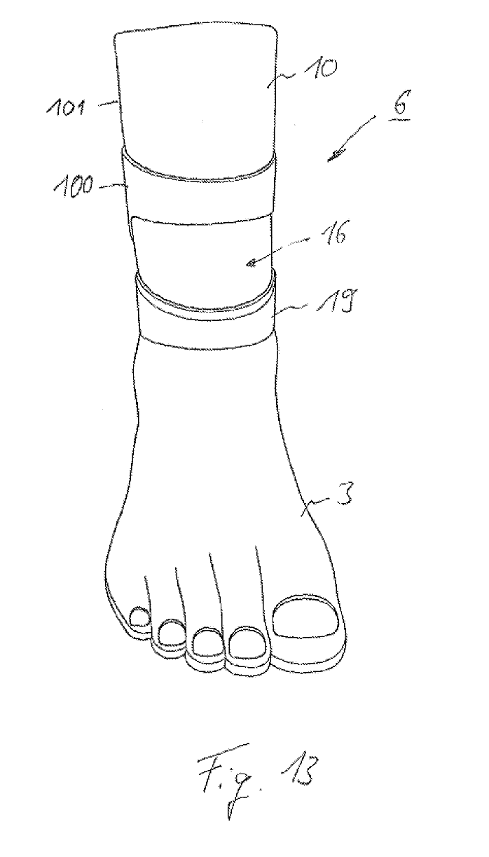

FIG. 14 shows an individual partial representation of a variant;

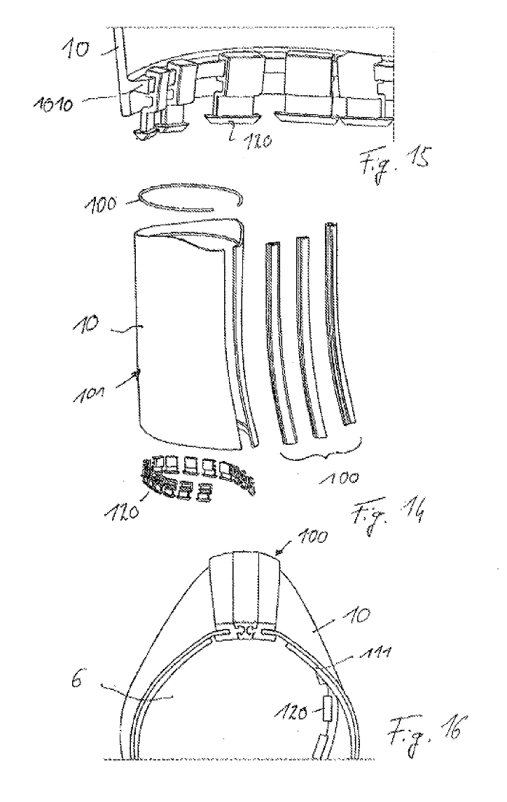

FIG. 15 shows a view of a detail of the variant according to FIG. 14;

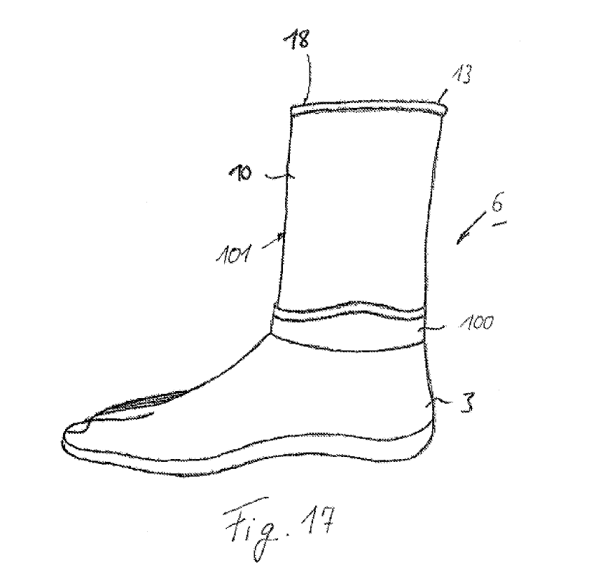

FIG. 16 shows a partial sectional representation of the variant according to FIG. 14 in an oblique plan view; and

FIG. 17 shows a variant of the invention in side view.

In FIG. 1a there is shown a base material 1, which may take the form of a plastic, foam, 3D knit or else a metal sheet. A blank 10.1 has already been partially cut out from the base material 1, which lies flat on an underlying surface; the contour of the part of the blank that has not yet been cut out is identified by a dashed line. The blank 10.1 is designed for the forming of a cosmetic lower leg cover and has a proximal end edge 13 and a distal end edge 14. In the completed state, the distal end edge 14 is assigned to a prosthetic foot, while the proximal end edge 13 is assigned to a prosthetic knee joint. From the end edges 13, 14 there extend a number of cutouts 31, 41 in the direction of the interior of the blank 10.1. From the proximal end edge 13, the cutouts 31 extend substantially in the distal direction, while the cutouts 41 from the distal edge extend substantially in the proximal direction. The form of cutouts 31, 41, which are shown as tapering away from the respective edge 13, 14, defines the form of the hollow body that is later formed therefrom.

Apart from the distal and proximal end edges 13, 14, lateral edges 11, 12 are provided, formed in a way corresponding to one another, so that after the bending of the blank 10.1 about a longitudinal axis, which runs substantially from proximal to distal, they are opposite one another and run along the longitudinal extent of the prosthesis element.

In Figure lb there is shown a blank 10.1 in a further cut-out and bent state. It can be seen that the edges of the clearances 31, 41 have already been moved toward one another. As a result, after the corresponding edges that lie opposite one another in the respective cutout 31, 41 have been connected, a substantially closed surface area of the blank 10.1 is obtained. When the lateral edges 11, 12 lie opposite one another after further bending together of the blank 10.1, these are also connected to one another, so that a substantially closed cross section perpendicular to the longitudinal extent, that is to say from the proximal end edge 13 to the distal end edge 14, is obtained.

In FIG. 2 there is shown the base material 1 and a blank 10.1 for an upper leg prosthesis element, which is formed in particular as a lining or as a socket element. Here, too, there are distal and proximal end edges 13, 14. In the exemplary embodiment represented, only portions extending from the proximal end edge 13 are provided, in order to form a curved, substantially barrel-shaped form of the prosthesis element after the lateral edges 11, 12 have been joined to one another. In FIG. 2, a cutting line 21, along which the blank 10.2 can be separated from the base material 1, is represented as a dash-dotted line; the blank 10.2 is preferably cut out, for example by using a laser beam, a water jet, a heated wire, a knife or shears. Other separating methods are also possible to separate the blank 10.2 from the base material 1.

FIG. 3 shows a third blank 10.3 in the form of a foot connection. A number of cutting lines 21, 22, 23 for blanks 10.3 of different sizes are provided on the base material 10, so that an orthopedic technician can separate the blank for different sizes from the base material 1 in an easy way.

In FIG. 4, the three prosthesis elements that are shown as blanks in FIGS. 1 to 3 and have been explained are represented in the finished form. The upper leg lining comprising the blank 10.2 has a closed cross section; the edges formed by the cutouts 31 are connected to one another, so that a closed cross section is obtained. The tapering and curved cutouts in the direction of the opposing end edge have the effect of achieving the barrel-shaped contour tapering toward the end edges.

The middle prosthesis element from the blank 10.1 is formed as a lower leg part and has a contour that corresponds to the natural contour of a lower leg. At the distal end edge 14, the foot connection is arranged on the blank 10.3, preferably securely fastened thereto, for example by welding or adhesive bonding, so that a complete cosmetic lower leg cover that adjoins the prosthetic foot is obtained. It is also possible in principle, if an appropriate material is chosen, that the prosthesis elements produced from the blanks 10.1, 10.2 and 10.3 also take the form of load-bearing prosthesis elements.

In FIG. 5 there is shown a variant of Figure lb in which a form-fitting element 5 in the form of a profiled strip is provided at the lateral edge 12. The form-fitting element 5 extends over the entire length of the edge 12 and has a recess, in which the blank 10.1 is received and secured in the region of the edge 12. The securement on the blank 10.1 may take place in a clamping or form-fitting manner. On the side of the blank 10.1 opposite from the left-hand edge 12 in the drawing, the edge 11 that has not yet been inserted into the form-fitting element 5 can be seen; the form-fitting element 5 has on the side opposite from the already inserted edge 12 a recess that is formed in a way corresponding to the recess for receiving the already inserted edge 12, so that the right-hand lateral edge 11 merely has to be inserted into this recess to assemble and close the blank 1 along the lateral edges 11, 12. There, too, the blank 10.1 is held in a clamping or form-fitting manner.

FIG. 6 shows a variant of the fastening and assignment of the lateral edges 11, 12 of the blank 10.1 to one another in a sectional representation. Arranged on the upper side and similarly on the underside of the blank 10.1 are velcro fasteners 5, which bridge the join between the lateral edges 11, 12 and can be used for the form-fitting fixing of the edges 11, 12 in relation to one another. Fleece areas are advantageously provided or formed on the outer side and/or the inner side of the blank 10.1, so that the hook areas of a velcro fastener 5 can engage directly. As an alternative to the provision of fleece areas, it is possible that the surface of the material of the blank allows the engagement of the hook areas of the velcro fasteners 5, so that a direct form-fitting locking of the hook areas of the velcro fasteners 5 with the surface of the blank 10.1 can take place. All that is then necessary for closing the blank 10.1 is to place strips of velcro fasteners or hook areas of velcro fasteners 5 over the join of the opposing lateral edges 11, 12 and anchor them there. The velcro fasteners may extend along the longitudinal extent of the join and be dimensioned so as to extend over the entire length; alternatively, the orientation of the velcro fasteners may run perpendicularly or diagonally in relation to the longitudinal extent of the join between the two lateral edges 11, 12.

A variant of the securement of the two edges 11, 12 to one another and in relation to one another is represented in FIG. 7, in which the clamping profile according to FIG. 5 is shown in a cross-sectional representation. In the position represented, the form-fitting element 5 is formed as an H lying on its side, and has a vertical connecting web 51 and two horizontally running extension arms 52, which extend on both sides of the connecting web 51. Projections 53 may be formed at the ends of the extension arms 52, and be formed so as to be prestressed onto the material of the blank in order to hold the blank 10.1 securely between them. The connecting web 51 substantially corresponds in its length to the material thickness of the material of the blank, so that the lateral edges 11, 12 can be inserted completely into the receiving space formed between the connecting web 51 and the extension arms 52. The extension arms 52 prevent displacement of the edges 11, 12 of the blank 10.1 parallel to the longitudinal extent of the connecting web 51; the projections 53 prevent displacement of the edges 11, 12 to one another or away from one another. If the bank 10.1 has not been inserted into the form-fitting element 5, the distance between the projections 53 may be less than the material thickness of the blank 10.1; the extension arms 52 are advantageously elastically formed and, after the insertion of the blank 10.1 into the form-fitting element 5, bring about prestressing and clamping retention.

In addition or as an alternative to this clamping retention, it is possible that there are within the blank 10.1 recesses corresponding to the projections 53, so that a form-fitting locking that acts in the direction of extraction or direction of insertion of the blank 10.1 can take place at least over part of the overall length of the form-fitting element 5. The extension arms 52 with the projections 53 directed in the direction of the blank 10.1 may extend over the entire length of the form-fitting element 5; as an alternative to this, a configuration of the form-fitting element 5 in which only a portion has the extension arms 52 and the projections 53 may be provided.

In FIG. 8 there is shown an overall view of a prosthesis or prosthesis device 2, which has a prosthetic foot 3, a lower leg part 4 fastened to the proximal end of the prosthetic foot 3 and a fastening device 50 arranged at the proximal end of the lower leg part 4. The fastening device 50 serves for mounting the prosthesis device 2 on an upper leg socket that is not represented. The fastening device 50 is mounted in an articulated manner on the lower leg part 4, and consequently part of a prosthetic knee joint. Arranged above the prosthetic foot 3, that is to say adjoining the proximal end of the prosthetic foot 3, is a prosthesis element 6 in the form of an ankle cuff, which has a main body 101. The ankle cuff 6 covers over the transitional region from the prosthetic foot 3 to the lower leg part 4 and extends approximately over one third of the length of the lower leg part 4. The ankle cuff 6 completely surrounds the lower leg part 4 and is reversibly secured on the prosthetic foot 3 by way of form-fitting elements that are not represented.

In FIG. 9, the prosthesis device 2 with the ankle cuff 6 is shown obliquely from the rear. In the lower leg part 4, damping devices and an electronic control unit as well as the prosthetic knee joint can be seen. The ankle cuff 6 itself has on the posterior side a stiffening element 100, which is arranged on the main body 101, and is arranged on the posterior side of the prosthesis device 2, that is to say in the region of the calf, and extends substantially in the longitudinal direction of the lower leg part 4. The stiffening element 100 serves on the one hand for ensuring the stability of the ankle cuff 6 in its longitudinal extent and, during the use of the ankle cuff 6, prevents it from slipping down from the lower leg part 4 in the direction of the prosthetic foot 3, and possibly becoming folded. The stiffening element 100 serves on the other hand for connecting the opposing ends or lateral edges of the blank 10 from which the main body 101 is formed. In the exemplary embodiment represented, the main body 101 is not formed as a circular or oval material blank with a closed cross section, but as a flat blank with an open cross section, the opposing edges 11, 12 of the blank 10 being connected to one another by way of the stiffening element 100, which at the same time performs the function of the form-fitting element 5, so that a hollow space for receiving the prosthesis device 2, in particular the lower distal part of the lower leg part 4, is provided.

In FIG. 10 there is shown a variant of the prosthesis element as an ankle cuff 6, which is arranged on a lower leg part 4. The prosthetic foot is not represented in FIG. 10. The ankle cuff 6 provides a main body 101 with a blank 10 of a flexible material, in particular a textile, a foam, or a combination of a number of flexible materials. In the exemplary embodiment represented, the main body 101 is formed in the manner of a flexible tube and has a closed cross section, which has been produced by connecting opposed edges of the blank 10. Arranged on the outer side of the main body 101 is a stiffening element 100 in the form of a frame, which forms inter alia the peripheral, distal end of the ankle cuff 6. Fashioned at the distal end of the stiffening element 100 are fastening elements 120 for the form-fitting securement on the prosthetic foot. Apart from the one-piece variant represented, with formed-on fastening elements 120, it is possible to secure separate fastening elements on the stiffening element 100, in order then to bring these into engagement with form-fitting elements on the prosthetic foot and to bring about form-fitting securement there of the ankle cuff 6 on the prosthetic foot.

At the distal end of the main body 101, the stiffening element 100 is formed as a peripheral closed ring, from which there extends from the posterior end, which is formed on the right in FIG. 10, an obliquely upwardly running frame portion 145. The transition from the lower, annular end to the upwardly pointing frame portion 145 is formed as a hinge device 140, so that a resilient movement of the anterior, or front, region of the stiffening element 100 downward or in the distal direction can take place. Provided for this purpose on the stiffening element 100 is an anterior clearance 16, which is filled by the material of the main body 101.

From the obliquely forwardly extending frame portion 145 of the stiffening element 100 there extends, slightly upwardly inclined, an upper frame portion 135 obliquely to the rear and upward, that is to say toward the proximal end of the ankle cuff 6, and thus forms a clip, which extends around the posterior part of the main body 101 above the posterior hinge 140. Arranged and formed at the transition from the obliquely forwardly pointing frame portion 145 to the obliquely rearwardly pointing frame portion 135 is a second hinge element or a second hinge device 130, so that the obliquely rearwardly extending frame portion 135 can be displaced downward. Formed underneath the upper frame portion 135 is a posterior clearance 15, which is likewise closed by the material 1 of the main body 101. If a plantar flexion of the prosthetic foot 3 is performed, there is obtained by analogy with the natural movement of a foot a displacement of the upper, rearwardly pointing frame portion 135 about the hinge device 130 and a flexible deformation of the material 1 of the main body 101 within the clearance 15 due to a compression, while the flexible material 1 of the main body 101 in the region of the anterior clearance 16 is stretched and the forwardly extending frame part 145 is displaced upward. In the case of a dorsal flexion, the compression takes place in the region of the anterior clearance 16 and stretching takes place in the region of the posterior clearance 15. Apart from a one-part configuration of the stiffening element 100, it is envisaged that it is constructed in a multi-part or modular manner, so that for example the lower, annular frame is connected to the upper frame portions, possibly connected in an articulated manner by way of a hinge or a number of hinges.

FIG. 11 shows in a side view the embodiment according to FIG. 10 in the state mounted on the prosthetic foot 3. The fastening elements 120 have been inserted into recesses in the upper edge of the end of the prosthetic foot that are not represented and allow flush mounting of the ankle cuff 6 on the prosthetic foot 3 to be carried out. Apart from reliable securement against an extraction movement, that is to say away from the prosthetic foot 3, the fastening elements 120 also bring about adaptation of the ankle cuff 6 to the contour of the respective prosthetic foot 3, since, apart from the main body 101, the stiffening element also has flexibility and elasticity, so that different prosthetic feet 3 can be provided with just one ankle cuff 6. It can be seen in FIG. 11 that the stiffening element 100 keeps the main body 101 in the desired form, so that the main body 101 encloses a hollow space and protrudes beyond the proximal edge of the stiffening element 100. On the inner side, a friction-reducing coating 18 may be arranged at the proximal edge 13 of the main body 101, in order to facilitate relative movement between the lower leg part 4 that is not represented and the material of the main body 101.

FIG. 12 shows the embodiment according to FIG. 11 in a rear view; the prosthetic foot 3 with the peripheral stiffening element 100 finishing flush at the circumference can be seen. In the posterior region, that is to say in the region of the Achilles heel, the hinge device 140, which is formed by the clearance 16, is formed above the distal ring of the stiffening element 100. The hinge device 140 allows the part or portion of the stiffening element 100 that is extending upward, that is to say toward the proximal end of the ankle cuff 6, to be easily flexed; in the case of the posterior hinge 140, this means that, in the event of a dorsal flexion, the upwardly adjoining portions of the stiffening element 100 can be displaced forward and downward, that is to say in the direction of the prosthetic foot. In the case of a plantar flexion, the upper or anterior hinge 130 undertakes this function and allows the posterior, proximal portion of the stiffening element 100 to be displaced in the distal direction, so that the posterior clearance 15 is reduced in size and the material of the main body 101 is compressed.

FIG. 13 shows the embodiment according to FIGS. 11 and 12 in a frontal view. It can be seen that the stiffening element 100 has in the anterior region a clearance 16, which is filled by the main body 101. The stiffening element 100 has in the distal region a peripheral ring 19 with a closed cross section. The stiffening element 100 serves for increasing the intrinsic stability of the ankle cuff 6 and at the same time provides mechanical protection against environmental influences for the ankle joint or the electronic unit arranged in the lower leg part 4. The stiffening element 100 may be welded, adhesively bonded, sewn or molded on the material 1 of the main body 101. In the exemplary embodiment represented, the stiffening element 100 is arranged on the outer side of the main body 101; there is also the possibility that it is arranged on the inner side of the main body or includes the main body 101, that is to say is arranged on the outer side and the inner side.

A variant of the invention that shows an embodiment according to FIG. 8 an individual partial representation is represented in FIG. 14. The main body 101 is configured as a substantially flat blank 10 and is formed in such a way that the opposing edges can be joined together in order to form a hollow body similar to a flexible tube. In the exemplary embodiment represented, the stiffening element 100 is of a multi-part form and provides two side rails, which are secured on the main body 101. The side rails are connected to one another in a form-fitting manner by way of a central rail. For this purpose, the central rail has recesses, in which form-fitting elements of the side rails engage. In order to achieve circumferential stability, a further stiffening element 100 is provided, arranged at the proximal end of the main body 101 in the exemplary embodiment represented. The second stiffening element 100 may be formed as a resilient wire or spring clasp.

Provided at the distal end of the main body 101 are separate fastening elements 120, which are secured there in a form-fitting manner and have projections by which the fastening elements 120, and consequently also the main body 101, can be secured with the fastening element 100 on the prosthetic foot.

FIG. 15 shows an enlarged view of a detail the inner side of the distal end of the main body 101, arranged on which is a peripheral projection 1010, which may for example be molded thereon or formed thereon. The projection 1010 has a V-shaped cross section, which tapers toward the outer wall of the main body 101. The fastening elements 120 have a dovetail-like recess, corresponding to the form of the projection 1010, so that they can engage in a form-fitting manner in the projection 1010. Formed at the distal end of the fastening elements 120 are arrow-like projections, in order to allow form-fitting locking by way of the engagement with corresponding recesses in the prosthetic foot. The fastening elements 120 are reversibly and displaceably secured on the main body 101, so that different numbers of fastening elements 120 may be secured on the respective blanks. The displaceability of the fastening elements 120 on the main body 101 makes it possible to cater for different sizes of prosthetic feet with the blank of the main body 101 or, possibly with slight modifications, to allow an adaptation. The larger the prosthetic feet are, the greater the distance between the individual recesses for the respective fastening elements, so that an adaptation cannot be freely performed without displaceable fastening elements 120.

FIG. 16 shows the fully assembled ankle cuff 6 with the main body 101, in the stiffening element 100 acting in the circumferential direction and also the stiffening element 100 acting in the longitudinal extent, with the two outer rails and the middle rail secured thereon in a form-fitting manner. The stiffening elements 100 have the effect of forming a closed hollow space 6, which receives the respective component of the prosthesis device.

Apart from the application as an ankle cuff, the prosthesis element 6 may also be secured on other prosthesis components and, apart from covering functions, also undertake load-bearing and structural tasks.

In FIG. 17 there is shown a further variant of the invention. The variant corresponds substantially to the embodiment according to FIG. 11, but the stiffening element 100 is arranged or formed in the distal region of the main body 101, for example by molding on, forming on or adhesively attaching a separate layer of material or by thickening, and possibly changing the properties, of the base material 1 in order to achieve increased dimensional stability in the distal region of the ankle cuff 6. The proximal portion of the main body 101, that is to say the portion that is arranged proximally in relation to the stiffening element 100, has sufficient intrinsic stability to bridge the intermediate space between the prosthetic foot 3 and the lower leg part 4 that is not represented. The change in the properties of the material 1 of the main body 101 in the event of thickening may take place by compression or thermal treatment. Apart from a separate configuration of the stiffening element 100, it is also possible in principle in the case of the other embodiments to configure them or it as a thickening or thickenings.

1. A prosthesis element for lining a prosthesis or forming a prosthesis component, the prosthesis element comprising:
at least one blank formed from a flat base material, the at least one blank being bent to form a hollow body;
wherein opposing edges of the at least one blank are connected to one another when the at least one blank is bent to form the hollow body.

2. The prosthesis element as claimed in claim 1, wherein the base material consists of a foam, a 3D knit, a plastic or a metal.

3. The prosthesis element as claimed in claim 1, wherein the at least one blank includes a plurality of blanks formed from the base material, the plurality of blanks being connected to one another.

4. The prosthesis element as claimed in claim 1, wherein the blank (10) or theplurality of blanks are at least one of adhesively bonded or welded together.

5. The prosthesis element as claimed in claim 1, further comprising a form-fitting element arranged on the at least one blank and configured to connect the opposing edges to one another.

6. The prosthesis element as claimed in claim 1, wherein the at least one blank has a proximal end edge and a distal end edge, and-from the distal end edge there extends at least one cutout in the a proximal direction or from the proximal end edge there extends at least one cutout in a distal direction.

7. The prosthesis element as claimed in claim 6, further comprising devices for securement on the prosthesis are arranged or formed on the proximal end edge or the distal end edge.

8. The prosthesis element as claimed in claim 1, further comprising a number of cutting lines for blanks of different sizes provided on the base material.

9. The prosthesis element as claimed in claim 1, wherein the prosthesis element is formed as a cosmetic prosthesis cover or as a load-bearing structural component.

10. The prosthesis element as claimed in claim 1, further comprising at least one stiffening element arranged on the main body to increase an intrinsic stability of the prosthesis element.

11. The prosthesis element as claimed in claim 10, wherein the stiffening element comprises a material that has a greater deformation resistance than a deformation resistance of the base material.

12. The prosthesis element as claimed in claim 10, wherein the stiffening element is formed as a frame which surrounds a main body formed from the base material.

13. The prosthesis element as claimed in claim 10, wherein at least one hinge device is arranged in the stiffening element to facilitate flexion about a joint.

14. The prosthesis element as claimed in claim 10, wherein the prosthesis element is symmetrically formed medial-laterally.

15. The prosthesis element as claimed in claim 10, wherein the stiffening element forms at least one of a distal end or a proximal end of the prosthesis element.

16. The prosthesis element as claimed in claim 10, wherein the stiffening element comprises cutouts.

17. The prosthesis element as claimed in claim 10, wherein the stiffening element closes a cross section of the blank.

18. The prosthesis element as claimed in claim 10, further comprising fastening elements configured to provide form-fitting securement of the prosthesis element on a prosthetic foot, the fastening element being arranged on the stiffening element.

19. The prosthesis element as claimed in claim 1, further comprising a friction-reducing coating arranged on a proximal inner side of the base material.

20. The prosthesis element as claimed in claim 18, further comprising fastening elements arranged on the base material and configured to provide form-fitting securement of the prosthesis element on the prosthetic foot.

21. A method for producing a prosthesis element from a flat base material, the method comprising:
cutting out at least one blank from the flat base material;
bending the at least one blank to form a hollow body;
connecting two edges of the at least one blank that lie opposite one another after the bending.

22. The method as claimed in claim 21, wherein the at least one blank is configured to bend around a prosthesis and the two edges are subsequently connected to one another.

23. The method as claimed in claim 21, wherein the at least one blank is cut out along cutting lines previously provided on the base material.

24. The method as claimed in claim 21, wherein the two edges are reversibly connected to one another or adhesively bonded or welded together.

25. The method as claimed in claim 21, wherein the at least one blank includes a plurality of blanks connected to one another to form the hollow body.
